(19) United States
(12) Patent Application Publication
(10) Pub. No.: US 2007/0225312 A1
(43) Pub. Date: Sep. 27, 2007

(54) TERGURIDE / PROTERGURIDE FOR THE TREATMENT OF CHRONIC PAIN

(75) Inventors: GEORG LUDWIG, ESCHEN (LI); REINHARD HOROWSKI, BERLIN (DE); HARALD BLIESATH, ALLENSBACH (DE); RUDOLF REITER, APPENZELL (CH)

Correspondence Address: J C PATENTS, INC., 4 VENTURE, SUITE 250, IRVINE, CA, 92618, US

(73) Assignee: ERGONEX PHARMA GMBH, APPENZELL (CH)

(21) Appl. No.: 11/689,355

(22) Filed: Mar. 21, 2007

(30) Foreign Application Priority Data
Mar. 21, 2006 (DE) 10 2006 013 307.2

(51) Int. Cl. A61K 31/48

(52) U.S. Cl. 514/288

(57) ABSTRACT

The present invention relates to the use of terguride and proterguride for the prophylaxis and/or the treatment of chronic pain conditions as well as to pharmaceutical compositions comprising terguride and/or proterguride optionally together with an opiate analgesic.

### CROSS-REFERENCE TO RELATED APPLICATION

This application claims the priority benefit of German application serial no. DE 10 2006 013 307.2, filed Mar. 21, 2006. All disclosure of the German application is incorporated herein by reference.

The present invention relates to the use of terguride and proterguride for the prophylaxis and/or the treatment of chronic pain conditions as well as to pharmaceutical compositions comprising terguride and/or proterguride optionally together with an opiate analgesic.

Acute pain serves as alarm signal for the organism and leads to fast prevention and protection reactions. Intensive acute pain stimuli can cause persistent functional and structural changes within a short time which change the stimulus transmission and treatment in a persistent manner. Chronic pains without any evident value are the consequence.

If a pain event lasts for more than three to six months, it is referred to as chronic pain. Causes thereof may be incurable diseases such as malignant tumors or rheumatic diseases. However, the connection between the pain and the disorder or respectively the disease which originally caused the pain is often no longer identifiable or the original disorder can no longer be remedied. Furthermore, various environmental influences like stress or weather changes can trigger or enhance the pain. A chronic pain presentation often includes different forms of pain.

Back pains (amongst others as a consequence of herniated discs, nerve root compression syndrome), head pains (amongst others migraine, tension-type headache, cluster headache), rheumatic pains (amongst others arthritis, fibromyalgia), neuralgias (amongst others trigeminal neuralgia, herpes zoster), tumor associated pains (amongst others brain tumor, bone metastases), degenerative pains (amongst others osteoporosis, arthrosis) and phantom pains (amongst others after amputation, plexus lesion) are mentioned as the most frequent forms of chronic pain.

Chronic pains often last for several years or decades. Frequently, patients suffering from chronic pain develop emotional problems. Many pain patients suffer from inactivity and listlessness; they are hopeless and desperate, complain about feelings of anxiety and depression, perceive themselves as limited in their self-esteem. Such psychic symptoms are warning signs of a chronification, just as general, nonspecific physical complaints such as intestine associated problems (diarrhea or respectively constipation), irritable bladder, dizziness, dyspnea, palpitations or a feeling of tightness in the chest.

Different mechanisms in the peripheral and central nervous systems are involved in the causation of chronic pain. The sensitization of pain fibers and their local hyperexcitability are substantial pathogenic mechanisms which are relevant as far as peripheral pain perception in the course of the causation of chronic pain conditions is concerned. Other pathomechanisms comprise the longer lasting enhancement of pain signals and a recruitment of usually silent nerve fibers in the area of the spinal cord that lead to a larger spatial extension of the pain perception. Finally, in the brain the pain potentials arriving in increased number from the periphery lead to changes in the signal transmission in terms of an enhancement of the pain perception and a long-term change in pain processing.

Even when lasting only for a few minutes, intensive pain stimuli can lead to persistent structural and functional changes which intensify the transmission and the processing of pain stimuli. These procedures are similar to cellular activities such as those that can be observed in all more complex, neuronal learning processes; consequently, it is analogously referred to as pain memory. In said context, the term pain memory includes the ability of the nervous system to generate a memory trace for an occurred painful stimulation through the whole pain processing system.

In this context, certain nuclei in the brain have a key function in pain processing. The nucleus accumbens serves as central switching station for the transmission and interpretation of pain stimuli as far as ascending pain impulses from the body are concerned. In a similar manner, the nucleus raphe has a key role in the transmission and modulation of descending pain signals.

A large arsenal of drugs is available for the treatment of chronic pain: Nonsteroidal anti-inflammatory drugs (NSAIDs) and non-opiate analgesics are the most frequently used drugs for pain medication. NSAIDs have analgesic, anti-inflammatory and antipyretic characteristics and are used for the treatment of slight to moderate pains. For the treatment of moderate to severe pains, other non-opiate analgesics such as for example aspirin, paracetamol or COX-I/II inhibitors are frequently used in combination with opioids like oxycodone and hydrocodone. Opiates such as morphine, fentanyl, methadone, hydromorphone, oxycodone, hydrocodone and meperidine are used for the treatment of severe pains.

However, the representatives mentioned frequently only ensure a partial pain relief, they loose their efficacy after a short time and often are associated with significant side effects. In this context, particularly the NSAIDs as well as the opiates have to be mentioned. Both classes of active agents have severe side effects. Gastrointestinal disorders and ulcers, kidney damage and hypersensitivity reactions are described as side effects of the therapy using NSAIDs. In addition to gastrointestinal side effects such as obstipation, a series of effects mediated by the central nervous system like sleepiness, nausea, confusion, respiratory depression, loss of efficacy and physical or psychic addiction have to be mentioned as disadvantages of opiates with effect on the central nervous system such as, for example, morphine and oxycodone.

Thus, there more effective analgesic active agents having efficacy on an extensive spectrum of pain conditions, with minimal or no side effects and not leading to tolerances or to physical or psychic addiction are still required.

Thus, the object of the present invention is to provide a drug for the treatment of fibromyalgia and chronic pain conditions which efficiently combats the symptoms and has considerably less side effects as the drugs known in the state of the art.

This object is solved by the technical teaching of the independent claims. Other advantageous embodiments, aspects and details of the invention result from the dependent claims, the description and the examples.

The present invention discloses the use of terguride or proterguride or a combination of both for the manufacture of drugs for the prophylaxis and the treatment of chronic pain and chronic pain conditions.

Surprisingly it was found that terguride or proterguride have been particularly efficient in the treatment of chronic pain conditions in addition to the known effects of the active agents according to the invention on Parkinson's disease, restless legs syndrome and hyperprolactinemia. The effect is realized within a well acceptable dose range without any side effects or with only minor, transient side effects such as nausea, emesis and orthostatic disorders being caused. Furthermore, the absence of negative effects of the inventive substances on the profile and quality of sleep has to be mentioned, which allows for a treatment of chronic pain conditions with few side effects.

Particularly in case of persistent musculoskeletal pains and persistent visceral pains, terguride and proterguride have been shown to be very efficient.

Thus, according to the invention, the two compounds are suitable for the treatment of persistent back pains, back pains for example as a consequence of herniated discs or nerve root compression syndrome, persistent neck pains, persistent shoulder pains, persistent joint pains, rheumatic pains, arthritis, fibromyalgia and Chronic Fatigue Syndrome, wherein fibromyalgia is particularly preferred.

Chronic Fatigue Syndrome (CFS) which is also known as Chronic Fatigue or Post Viral Fatigue Syndrome indicates a paralyzing mental and physical exhaustion or respectively exhaustibility and other symptoms of exhaustion which are different in each individual. The exhaustion has to last at least for 6 months to be referred to as Chronic Fatigue Syndrome and leads to a severe performance reduction compared to the habitual former performance, a fact that prevents the patient from leading a normal live.

Furthermore, terguride and proterguride as well as combinations of both are very well suited for the prophylaxis and the treatment of head pains and migraine. Terguride or proterguride used in small doses in a prophylactic manner or in case of the first signs of head pains and migraine relieve these discomforts considerably and in some cases within some hours.

Furthermore, the substances terguride and proterguride are very well suited for the prophylaxis and the treatment of pains associated with premenstrual syndrome, mastalgia, of stomach pains associated with the irritable colon as well as pains associated with carcinoid syndrome. Also, degenerative pains, osteoporosis, arthrosis as well as phantom pains for example after amputation or plexus lesion are other types of pain which can be treated with terguride and/or proterguride. According to the invention, the two active agents can also be used for addressing pains or respectively pain conditions associated with neuralgias, trigeminal neuralgia, herpes zoster, postherpetic neuralgias as well as neuropathic pains and tumor associated pains. The tumor associated pains are particularly caused by brain tumors or bone metastases.

Another aspect of the present invention concerns a combination of terguride or proterguride or of terguride and proterguride with an opiate analgesic as well as to the use of this combination for the prophylaxis and the treatment of the indications mentioned herein.

It has been shown that a combination of terguride and at least one opiate analgesic or a combination of proterguride and at least one opiate analgesic or a combination of terguride and proterguride and at least one opiate analgesic is more efficient than the respective single components, with the undesired side effects of the at least one opiate analgesic being reduced or the activity of the at least one opiate analgesic being increased.

The following compounds can be particularly mentioned as opiate analgesics: dihydrocodeine, tramadol, morphine, morphine sulfate, oxycodone, methadone, hydromorphone, buprenorphine as well as fentanyl.

The applications according to the invention are suitable for a continuous application since no physical or psychic addiction has occurred and also in case of administration over several months, no loss of efficacy has been observed. Moreover, it could also be shown that both terguride and proterguride could be used with a comparable efficacy when the presentation was recurrent, even in cases where it was discontinued once the pain presentation had subsided.

The substances according to the invention are characterized by a broad spectrum of activity as far as the dopaminergic, serotoninergic and the noradrenergic neurotransmitter systems are concerned. In the active agents, one single molecule combines different active principles which are relevant for different central nervous pain transmission systems. It is supposed that precisely this combination of characteristics in the substances according to the invention is of crucial importance for the high therapeutic efficacy. Simultaneously, this combination has a positive effect on the mood, the cognitive performances and the daily activities of patients. A surprising, positive effect on the overall condition of patients, an improvement of the sleep quality as well as the good tolerance of the active agents are advantageous for the good compliance of patients and contribute considerably to the success of the therapy using the active agents according to the invention.

Another aspect of the present invention relates to pharmaceutical compositions containing at least one pharmacologically acceptable carrier, auxiliary agent and/or solvent in addition to terguride or proterguride or a combination of terguride and proterguride or respectively their pharmacologically acceptable salts.

Furthermore, it is preferred that the pharmaceutical composition further comprise at least one opiate analgesic. Together with terguride and/or proterguride, this at least one opiate analgesic can be contained in one single galenic formulation or it can be present as a second galenic formulation which can be applied independently of the formulation containing terguride and/or proterguride. Furthermore, the presence of two formulations in the pharmaceutical compositions is preferred since thus a certain concentration of terguride and/or proterguride regarding the opiate analgesic can be better adjusted or respectively changed or the opiate analgesic can be entirely discontinued.

These pharmaceutical compositions preferably contain terguride in the dose range of 0.1-3.0 mg per pharmaceutical formulation or proterguride in the dose range of 0.002-0.5 mg per pharmaceutical formulation. If the pharmaceutical formulation contains both terguride and proterguride, the dose ranges from 0.1-3.0 mg for terguride and from 0.002-0.5 mg for proterguride are preferred.

The pharmaceutical compositions are preferably provided in the form of pills, tablets, enteric-coated tablets, film tablets, layer tablets, prolonged release formulations for oral administration, sugar-coated tablets, suppositories, gels, creams, syrup, inhalation powders, granulates, emulsions, dispersions, microcapsules, microformulations, nanoformulations, liposomal formulations, capsules, enteric-coated capsules, powder, powder blends, microcrystalline formulations, inhalation sprays, drops, nose drops, nose sprays, aerosols, ampules, solutions, juices, suspensions, infusion solutions or injection solutions. Capsules, sugar-coated tablets, enteric-coated formulations, juices, suspensions, suppositories, solutions, injections and granulates are preferred.

Preferably, the pharmaceutical compositions are suitable for inhalation or for intravenous, intraperitoneal, intramuscular, intravaginal, intrabuccal, percutaneous, subcutaneous, mucocutaneous, oral, peroral, lumbar, rectal, transdermal, topical, intradermal, intragastric or intracutaneous administration.

For example, lactose, starch, sorbitol, sucrose, cellulose, magnesium stearate, dicalcium phosphate, calcium sulfate, talc, mannitol, ethyl alcohol and the like can be used as pharmacologically acceptable carriers. Powders as well as tablets can be composed of such a carrier to between 5 and 95%.

Furthermore, starch, gelatin, natural sugars, natural as well as synthetic gums such as, for example, acacia gum or guar gum, sodium alginate, carboxymethyl cellulose, polyethylene glycol and waxes can be used as binders. Boric acid, sodium benzoate, sodium acetate, sodium chloride and the like can serve as lubricants.

Furthermore, disintegrants, colorants, flavors and/or binders can be added to the pharmaceutical compositions.

Fluid formulations comprise solutions, suspensions, sprays and emulsions, such as, for example water based or water-propylene glycol based injection solutions for parenteral injections.

For the preparation of suppositories, low melting waxes, fatty acid esters and glycerides are preferably used.

Capsules are made of, for example, methyl cellulose, polyvinyl alcohol or denatured gelatin or starch.

Starch, sodium carboxymethyl starch, natural and synthetic gums, such as, for example, carob flour, karaya, guar, tragacanth and agar as well as cellulose derivatives such as methylcellulose, sodium carboxymethylcellulose, microcrystalline cellulose as well as alginate, clay minerals and bentonites can be used as disintegrants. These components can be used in amounts from 2 to 30% by weight.

Sugar, starch derived from corn, rice or potatoes, natural gums such as acacia gum, gelatin, tragacanth, alginic acid, sodium alginate, ammonium sodium alginate, methylcellulose, sodium carboxymethylcellulose, hydroxypropyl methylcellulose, polyvinyl pyrrolidone as well as inorganic compounds like magnesium-aluminium-silicate can be added as binders. The binders can be added in amounts from 1 to 30% by weight.

Stearates such as magnesium stearate, calcium stearate, potassium stearate, stearic acid, high melting waxes as well as water-soluble lubricants such as sodium chloride, sodium benzoate, sodium acetate, sodium oleate, polyethylene glycol and amino acids such as leucine can be used as lubricants. Such lubricants can be used in amounts from 0.05 to 15% by weight.

### EXAMPLES

### Example 1

### Application of Terguride in the Case of Fibromyalgia

A 36 year-old patient suffering from fibromyalgia was treated with terguride.

The patient complained about chronic pain in the extremities as well as about stomach pains and continuous exhaustion and tiredness. The discomforts had persisted for several months. No pretreatment had been realized prior to the treatment with terguride.

The patient was treated with a daily dose of 3 mg of terguride which was administered in the morning and in the evening in single doses of 1.5 mg. The treatment was realized gradually. Over a period of 2 weeks, the daily dose of initially 0.25 mg was progressively increased to 3 mg.

The treatment was realized over a period of 25 weeks and the condition of the patient was examined weekly. After just 10 weeks, the condition had visibly improved, in particular regarding the pains in the extremities and the stomach pains recorded by means of a visual analog scale.

After 17 weeks, it was also nearly impossible to detect the chronic conditions of tiredness and exhaustion and according to the patient her normal performance, just as before the onset of the disease, was re-established.

### Example 2

### Application of Proterguride in the Case of Fibromyalgia

A 29 year-old female patient suffering from fibromyalgia was treated with proterguride.

The patient complained about stomach problems, stomach pains, pains in the chest as well as about swollen breasts and continuous exhaustion and tiredness. The discomforts had persisted for approx. one year. No pretreatment had been realized prior to the treatment with proterguride.

The patient was treated with a daily dose of 0.5 mg of proterguride which was administered in the morning and in the evening. The treatment was realized gradually. Over a period of 2 weeks, the daily dose of initially 0.05 mg was progressively increased to 0.5 mg.

The treatment was realized over a period of 33 weeks and the condition of the patient was examined weekly. After just 12 weeks, the condition had improved, particularly regarding the pains in the chest. Furthermore, a reduction of the swelling could be observed.

After 20 weeks, also the chronic conditions of tiredness and exhaustion as well as the stomach pains had largely disappeared and after 30 weeks, the patient was in the same condition as before the onset of the disease.

### Example 3

### Application of Terguride in the Case of Mastalgia/Premenstrual Syndrome

A 34 year-old patient suffering from mastalgia and stomach pains due to premenstrual syndrome was treated with terguride. Laboratory analyses showed that the woman's blood levels of prolactin were in the normal range. The therapy started in the middle of the cycle. Twice a day, a tablet containing 0.5 mg terguride was administered to the patient over a period of 3 months. The pain severity was observed by means of a visual analogue scale. During the therapy, the pains in the chest and stomach area decreased and the patient was able to renounce on the administration of additional pain medication. Furthermore, the patient reported a considerably improved general condition and joy of life. No side effects were observed. During the following months under therapy the patient remained symptom-free.

### Example 4

### Application of Terguride for the Prophylaxis of Migraine

A 65 year-old patient, suffering from migraine on average four times a month, was treated with terguride. The patient was treated with a daily dose of 2 mg terguride which was administered in the morning and in the evening in single doses of 1 mg. The treatment was realized gradually. Over a period of 2 weeks, the daily dose of initially 0.25 mg was progressively increased to 2 mg. The patient was treated over a period of 24 weeks. The frequency, duration and severity of the migraine attacks were recorded by means of a diary. Within 4 weeks following the start of the treatment, a reduction of 40% of the frequency of migraine attacks could be shown, as well as a reduction of the attack duration from cumulative 12 days before the therapy to 8 days under terguride. The consumption of pain medication under therapy with terguride was reduced by 46%. No side effects of the terguride therapy were observed. The patient reported an improvement of his general condition.

### Example 5

### Diabetic Neuropathy

About 15 months ago, a diabetic neuropathy was clinically diagnosed in a 56 year-old female patient who has been suffering from Type 2 Diabetes for 10 years. The patient was treated with 3×50 mg amitryptiline/day. At the time of the examination, the patient complained about pain conditions associated with burning sensations, shooting or stabbing pains and unpleasant formication, mainly in the feet and with increasing intensity during the night. On the numerical rating scale from 0=no pain to 10=maximum pain intensity, the patient reported mean pain values of 8-9 on average.

Under therapy with terguride (0.5 mg b.i.d.) the pain conditions and the general condition of the patient improved quickly (average pain value of 2-3 on the rating scale). No side effects of the terguride therapy were observed. No signs of an loss of efficacy under continuous treatment with terguride were observed.

### Example 6

### Postherpetic Neuralgia

A 65 year-old patient of normal weight complained about a severe burning sensation and pains in the dermal area of the chest. The anamnesis resulted in herpes zoster which, however, was only treated after the onset of the rash and the scab. The treatment of the pains with amitriptyline (3×50 mg/d) and subsequently with gabapentin (daily dose: 1500 mg) led only to a short-term improvement followed within 10 days by a considerable deterioration of the pains.

Under administration of terguride (0.5 mg t.i.d.) the pain presentation improved in a fast and continuous manner. No side effects were observed. After 3 months, the daily dose of terguride was gradually reduced in the frame of a treatment-free interval wherein the pain increased quickly.

### Example 7

### Tumor Associated Pain

A 62 year old tumor patient suffering from a pancreatic carcinoma complained about bone pains resulting from the development of bone metastases. The patient was treated with ibandronate. Additionally, the treatment was realized with ibuprofen, acetaminophen and desipramine. At short term, the treatment resulted in a pain relief which, however, decreased considerably within some weeks. After the start of the therapy with terguride (0.5 mg bid) the patient's pain conditions were reduced and the well-being improved continuously. No side effects were observed.

### Example 8

### Herniated Disc

Since two years, the patient (43 years) has been suffering from a herniated disc in the lower lumbar area (L5-S1) with extension to L4-L5. The chronic pain was treated with oxycodone of an initial dosage of 2×10 mg and the dose was increased to 2×40 mg per day. The pain intensity was considerably reduced under therapy. The longer the treatment lasted, the more complaints occurred due to constipation, finally requiring a dose reduction of oxycodone to 2×20 mg/day. This was related to an increase of the subjective pain intensity of the patient. Under co-therapy with terguride (2×0.5 mg/day) the patient was almost completely pain-free; also, the constipation symptoms had disappeared completely. Under chronic therapy, the therapy with terguride remained efficient.

### Example 9

### Migraine Prophylaxis

The 47 year-old patient suffered from migrainous headaches with an average of 4-5 attacks per month, lasting on average between 3 and 8 hours. For acute pain treatment during an attack, a dosage of 50 mg of sumatriptan was administered orally. For the purpose of migraine prophylaxis, the patient was treated with topiramate in a dosage of 200 mg per day. No reduction of the migraine activity was observed under medication. After 3 months, the medication was discontinued due to nausea and weight loss.

Under administration of terguride (2×0.25 mg per day), the number of pain attacks decreased considerably to only one pain attack within 6 weeks with low pain intensity; also, the well-being of the patient improved within 2 weeks after the start of the therapy. No side effects were observed. In the frame of a treatment-free interval, the treatment with terguride was discontinued, whereupon the migraine presentation returned.

1. Use of terguride or proterguride or a combination of terguride and proterguride or pharmacologically acceptable salts thereof for the preparation of a drug for the prophylaxis and/or the treatment of chronic pain or chronic pain conditions.

2. Use according to claim 1 for the treatment of persistent musculoskeletal pains and persistent visceral pains.

3. Use according to claim 2 for the treatment of persistent back pains, persistent neck pains, persistent shoulder pains, persistent joint pains and fibromyalgia.

4. Use according to claim 2 for the prophylaxis and the treatment of pains associated with premenstrual syndrome, mastalgia, stomach pain associated with irritable colon and pains associated with carcinoid syndrome.

5. Use according to claim 1 for the prophylaxis and the treatment of head pains and migraine.

6. Use according to claim 1 for the treatment of neuralgias, trigeminal neuralgia, postherpetic neuralgia, neuropathic pains and tumor associated pains.

7. Use according to claim 1 in combination with an opiate analgesic, dihydrocodeine, tramadol, morphine, morphine sulfate, oxycodone, methadone, hydromorphone, buprenorphine and fentanyl.

8. Pharmaceutical composition comprising terguride or proterguride or a combination of terguride and proterguride or their pharmacologically acceptable salts together with at least one pharmacologically acceptable carrier, auxiliary agent or solvent.

9. Pharmaceutical composition according to claim 8 suitable for inhalation or for intravenous, intraperitoneal, intramuscular, intravaginal, intrabuccal, percutaneous, subcutaneous, mucocutaneous, oral, peroral lumbar, rectal, transdermal, topical, intradermal, intragastric or intracutaneous administration.

10. Pharmaceutical composition according to claim 8 further comprising an active agent selected from the group comprising opiate analgesic, dihydrocodeine, tramadol, morphine, morphine sulfate, oxycodone, methadone, hydromorphone, buprenorphine and fentanyl.

11. Pharmaceutical composition according to claim 8 comprising terguride in the dose range of 0.1-3.0 mg per pharmaceutical formulation and/or proterguride in the dose range of 0.002-0.5 mg per pharmaceutical formulation.

12. Use according to claim 2 in combination with an opiate analgesic, dihydrocodeine, tramadol, morphine, morphine sulfate, oxycodone, methadone, hydromorphone, buprenorphine and fentanyl.

13. Use according to claim 3 in combination with an opiate analgesic, dihydrocodeine, tramadol, morphine, morphine sulfate, oxycodone, methadone, hydromorphone, buprenorphine and fentanyl.

14. Use according to claim 4 in combination with an opiate analgesic, dihydrocodeine, tramadol, morphine, morphine sulfate, oxycodone, methadone, hydromorphone, buprenorphine and fentanyl.

15. Use according to claim 5 in combination with an opiate analgesic, dihydrocodeine, tramadol, morphine, morphine sulfate, oxycodone, methadone, hydromorphone, buprenorphine and fentanyl.

16. Use according to claim 6 in combination with an opiate analgesic, dihydrocodeine, tramadol, morphine, morphine sulfate, oxycodone, methadone, hydromorphone, buprenorphine and fentanyl.

17. Pharmaceutical composition according to claim 9 further comprising an active agent selected from the group comprising opiate analgesic, dihydrocodeine, tramadol, morphine, morphine sulfate, oxycodone, methadone, hydromorphone, buprenorphine and fentanyl.

18. Pharmaceutical composition according to claim 9 comprising terguride in the dose range of 0.1-3.0 mg per pharmaceutical formulation and/or proterguride in the dose range of 0.002-0.5 mg per pharmaceutical formulation.

19. Pharmaceutical composition according to claim 10 comprising terguride in the dose range of 0.1-3.0 mg per pharmaceutical formulation and/or proterguride in the dose range of 0.002-0.5 mg per pharmaceutical formulation.

20. Pharmaceutical composition according to claim 17 comprising terguride in the dose range of 0.1-3.0 mg per pharmaceutical formulation and/or proterguride in the dose range of 0.002-0.5 mg per pharmaceutical formulation.
